(19) United States
(12) Patent Application Publication
(10) Pub. No.: US 2019/0099285 A1
(43) Pub. Date: Apr. 4, 2019

(54) NECK COLLAR FOR TREATMENT OF SLEEP APNEA

(71) Applicant: MORPHEUS MEDICAL SOLUTIONS, LLC., Duluth, MN (US)

(72) Inventors: Vance D. Bachelder, Duluth, MN (US); Jose Carrillo, III, Duluth, MN (US)

(73) Assignee: MORPHEUS MEDICAL SOLUTIONS, LLC., Duluth, MN (US)

(21) Appl. No.: 16/149,463

(22) Filed: Oct. 2, 2018

Related U.S. Application Data
(60) Provisional application No. 62/566,705, filed on Oct. 2, 2017.

(51) Int. Cl. A61F 5/56; A61H 9/00

(52) U.S. Cl. CPC A61F 5/56; A61H 9/0057; A61H 2201/5071; A61H 2201/5097; A61H 2201/1609; A61H 2201/5023; A61H 2201/165

(57) ABSTRACT

A collar for prevention of sleep apnea is disclosed. An illustrative collar includes upper and lower portions to provide a comfortable and generally air-sealed interface to the upper neck and jaw, and lower neck and chest, respectively, of the patient. In between, an intermediate section defines a chamber over the anterior neck to which negative pressure is applied, drawing tissue forward and away from the patient's airway, improving air flow for the patient and preventing sleep apnea.

### CROSS REFERENCE TO RELATED APPLICATIONS

The present application claims the benefit of and priority to U.S. Provisional Patent App. No. 62/566,705, filed Oct. 2, 2017 and titled NECK COLLAR FOR TREATMENT OF SLEEP APNEA, the disclosure of which is incorporated herein by reference.

### BACKGROUND

Millions of people suffer from obstructive sleep apnea (OSA). OSA is a condition where the upper airway collapses during sleep and significantly limits or obstructs air entry into the lungs. The mainstay of treatment for OSA is continuous positive airway pressure (CPAP). This works by applying positive air pressure to the upper airway which consists of the nasal passages, mouth, nasopharynx, oropharynx, and hypopharynx. CPAP pressure opens the upper airway, allowing the sleeper to breathe easily without intermittent obstruction and interruption of airflow into the lungs.

CPAP pressure is delivered via a mask applied over the nose (nasal mask) or over the nose and mouth (full face mask) with air pressure tubing running from the mask to a CPAP machine. A good mask seal is desirable as high leak rates from air escaping around the sides of the mask are uncomfortable and may disrupt sleep. High leak rates may also cause CPAP treatment to be ineffective.

Some sleepers find using a mask at night uncomfortable and so have difficulty sleeping with one. Further, some sleepers will easily fall asleep using a CPAP mask only to discover that sometime during the night it has come off or that they have removed it surreptitiously. These problems clearly make CPAP therapy less effective than it otherwise might be. Depending on how compliance is defined and the duration of study, compliance rates may be as low as 50% or less.

Oral appliances for sleep apnea are known as well. Such products pull the lower jaw forward during sleep to prevent airway obstruction. However, teeth may shift over time, causing changes to bite and various dental problems. Some patients may experience headaches or other physical discomfort as the oral appliance holds the jaw in an unnatural position. Again, depending on how compliance is defined and the duration of study, compliance rates may be as low as 50% or less.

Obstructive sleep apnea is associated with a number of health difficulties including ranging from daytime drowsiness to increased blood pressure. Thus there remains a need to provide additional therapies that may supplant or supplement those which are already known.

### Overview

The present inventors have recognized, among other things, that a problem to be solved is the desired for still further alternative treatments for sleep apnea. In an example, a collar is provided that is desired to maintain an open airway for the patient. In an example, negative pressure is applied via the collar to pull soft tissue away from the center of the throat, allowing for better breathing. Such negative pressure may be applied via an external apparatus such as a pump.

In some examples, the physical structure of the collar may aid in keeping the patient's jaw from dropping inferior and posterior relative to the chest, helping to avoid pharyngeal obstruction involving the tongue and soft palate. Such structural support, when combined with the positive atmospheric pressure seen by the nasopharynx, may also keep the soft palate in contact with the tongue, helping to keep both tongue and soft palate from obstructing the airway.

A first illustrative and non-limiting example takes the form of a collar for the treatment of sleep apnea comprising: a first end having a portion made of a soft, flexible material to provide a seal against the skin of the patient near the chin; a second end having a portion made of a soft, flexible material to provide a seal against the skin of the patient near the base of the neck; and a body between the first and second ends, the body having an inner wall for defining a chamber about the neck of the patient and at least one air channel associated with a first port in the inner wall.

Additionally or alternatively, the first and second ends may be configured such that the chamber is generally airtight to facilitate application of negative pressure to the neck of the patient to pull soft tissue away from the center of the throat, allowing the patient to breath.

Additionally or alternatively, at least one of the first or second ends may be made of silicone or a soft gel to facilitate patient comfort and a seal against the skin of the patient.

Additionally or alternatively, the body may comprise an inner layer of relatively more rigid material to maintain the chamber.

Additionally or alternatively, the first end may be configured to push open the soft palate by limiting movement of the jaw of the patient.

Additionally or alternatively, the body may include a back portion having a line of separation allowing the collar to be removed from and/or applied to the neck of a patient, and a front portion in which the first port is located.

Additionally or alternatively, the collar may further comprise a vent and a pressure sensor, the vent coupled to the air channel to allow release of pressure therein, wherein the pressure sensor is adapted to sense pressure in the chamber and/or air channel and open the vent to prevent over-pressurization.

Additionally or alternatively, the collar may further comprise a leak valve coupled to the inner wall and adapted to limit pressurization of the chamber.

Additionally or alternatively, the body may comprise a coiled support structure to allow longitudinal flexibility between the first and second ends, and lateral support to maintain the chamber.

Additionally or alternatively, the body may comprise a plurality of support structures in the form of rings, the rings being interrupted at the location of the line of separation.

Another example takes the form of system comprising a collar as in any of the preceding examples and an external vacuum motor coupled by a tube to a second port of the air channel.

Another example takes the form of a collar as in any of the preceding examples, further comprising a vacuum motor coupled to a second port of the air channel for applying a vacuum to the chamber.

A second illustrative non-limiting example takes the form of a method of treating a patient having a breathing disorder comprising: applying a collar to the patient's neck; and applying negative air pressure to the patient's neck via the collar.

In this second example, the breathing disorder may be obstructive sleep apnea.

A third illustrative non-limiting example takes the form of a method of treating a patient comprising making a collar to prevent sleep apnea by: obtaining one or more images of a region of the patient including the neck; and making a collar for treating sleep apnea using the obtained images to construct a collar having an upper end to provide an airtight and comfortable seal at the upper neck and/or jaw of the patient and a lower end to provide an airtight and comfortable seal at the base of the neck of the patient, with an intermediate section therebetween defining a chamber for applying negative pressure to the patient's anterior neck.

This overview is intended to provide an introduction to the subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a sagittal section view of the head and neck of a human;

FIG. 2 is an axial section of a human neck at the C4 vertebrae;

FIG. 3 is a side view of an illustrative device on a patient;

FIG. 4 is a rear view of an illustrative device with partial cut-away;

FIG. 5 is a side view of an illustrative system on a patient;

FIG. 6 is a side view of an illustrative integrated device on a patient;

FIG. 7 is a side view of an illustrative device construction;

FIGS. 8A-8B show a partial section view of a joint between layers of the embodiment of FIG. 7;

FIGS. 9-10 show a medial slice of each of two illustrative examples; and

FIG. 11 is a side view of another illustrative device.

### DETAILED DESCRIPTION

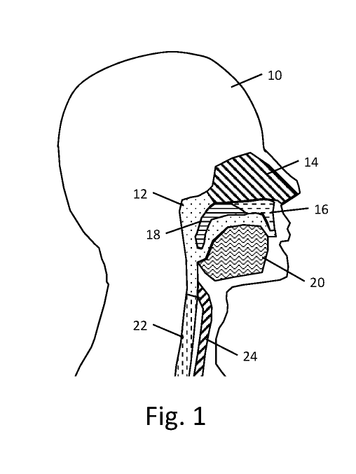

FIG. 1 is a sagittal section view of the head and neck of a human patient. Certain anatomy of interest is highlighted for the patient 10. The upper airway 12 can be seen extending up from the neck to the sinus cavity 14 and also through the mouth between the hard palate 16 and soft palate 18, and tongue 20. As the anatomy progresses inferiorly into the neck the esophagus 22 and trachea 24 divide, with the trachea 24 more anterior of the two.

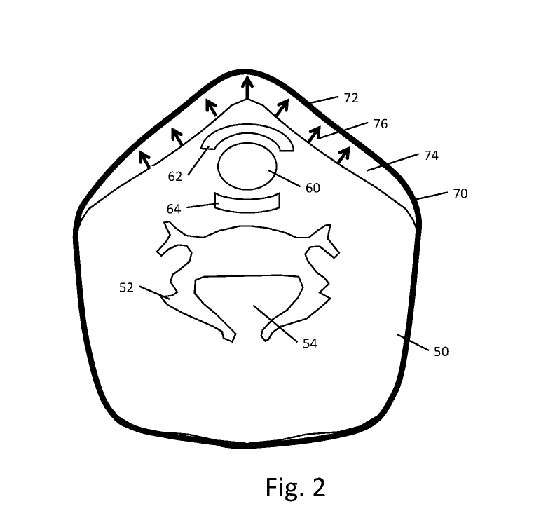

FIG. 2 is an axial section of a human neck at about the C4 vertebrae, with an illustrative device shown about the neck in order to illustrate the operation thereof. The neck 50 comprises the bony vertebral structure shown at 52 which defines the spinal column 54. Anteriorly of the bony vertebral structure 52 is the trachea 60, anterior of the esophagus 64, with the thyroid gland 62.

For some patients, excess tissue (typically fat) in the neck and or jaw region places pressure on the neck in certain positions, squeezing the trachea 60 and ultimately leading to certain forms of sleep apnea. Ordinary CPAP systems are designed to increase the air pressure within the trachea 60 (and other airways) in order to overcome the forces that cause sleep apnea, keeping the airway open. Some embodiments of the present invention, at least in part, avoid sleep apnea using a reversed approach.

As shown in FIG. 2, a device 70 may take the form of a neck collar that goes around the patient's neck. An anterior portion 72 of the device 70 is resilient, and is shaped to create a small cavity 74 over a portion of the anterior neck of the patient. Vacuum pressure is created in the cavity 74 to apply forces as shown at 76 that counteract the pressures that would otherwise close the airway 60.

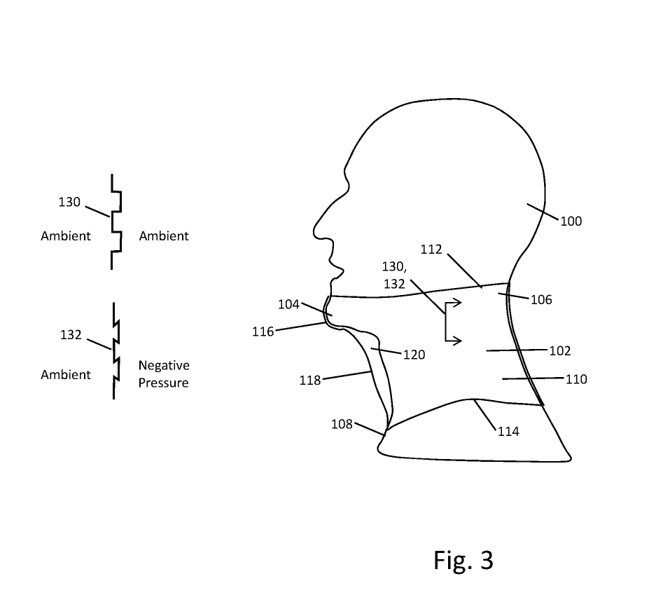

FIG. 3 is a side view of an illustrative device on a patient. The patient 100 is shown with neck 102, jawline 104, base of the skull 106, and upper chest 108. About the patient's neck 102 is an illustrative device in the form of a collar 110, which may be a neck collar similar in some respects to a cervical collar used for immobilizing the neck of a trauma patient, but with more flexibility and additional features discussed herein.

The collar 110 is configured for an air tight, or substantially air tight, seal against the patient's body at upper and lower ends thereof. A vacuum motor, which may be integral to the collar 110 or may be externally provided, causes negative pressure immediately around the neck and jaw, pulling the soft tissue away from the center of the throat to improve breathing, with a particular eye toward preventing the hypopneas and blood oxygen reductions associated with clinically significant sleep apnea. As noted above, the operation is the opposite of CPAP, which applies positive pressure inside the respiratory system of a patient, by applying negative pressure to areas of the patient that are linked to ease of breathing. In addition to the negative pressure, the collar 110 may support improved breathing by supporting the soft palette under the chin. The phrase “applying negative pressure” includes, in some examples, generating suction that pulls soft tissue.

The concept runs counter to the discussions of US PG Patent Application Publication No. 2007/0221231, which suggests that an adhesive may be used to pull on the neck. Use of adhesive, chronically, may become uncomfortable to the patient and may create points of strain and irritation at the edges thereof. Non-contact, negative air pressure is provided instead with some embodiments of the present invention. While adhesive may be used in some examples to aid in the creation of a seal, it is not the primary driver in affecting the soft tissue in the neck region in those embodiments that use adhesive.

Other prior devices have focused primarily on structural support, such as US PG Patent Application Publications No. 2003/0056785, and 2007/0256694 and/or a combination with electrical therapy and structural support as in US PG Patent Application Publication No. 2011/0230702. In some examples, an adhesive, such as a replaceable strip or strips of material permanently or temporarily coupled via adhesive, Velcro or snaps to the inside of the collar and having an adhesive such as that on tapes or bandages for application to the skin, may be used to aid in securing air-tightness or otherwise securing the collar on the patient. In other examples, no adhesive or strips/tape is used. Replaceable foam or gel pads, for example, may be provided at the upper and lower ends of the collar to achieve an air tight or mostly air tight seal.

Turning back again to FIG. 3, the collar 110 is placed over the neck of the patient, with the first or upper end 112 thereof extending generally over the jaw 104 of the patient with a flexible and elastic portion shown at 116. The upper end 112 extends as well to approximately the lower margin of the cranium generally at 106.

The second or lower end 114 of the collar 110 may generally reside near the upper chest of the patient. The lower end 114 may, for example, track the clavicle from the manubrium (or a location above the manubrium such as nearer the Adams Apple) generally to the lateral edge of the neck and then back to the top of the trapezius muscle group. The lower end 114 may, as shown, extend generally low along the back of the neck to a position about level with the Ti vertebra, though in other examples the lower end 114 may angle upward on the back of the patient to about the vertical middle of the neck (i.e., about C4 or C5, or higher or lower).

The upper end 112 and lower end 114 may be more pliable and softer than other regions of the collar 110. For example, a multilayer structure may be provided at the upper and/or lower ends 112, 114 with an outer layer of elastic material designed to achieve a hold, such as a stretchy fabric with elastic and inelastic fibers, while an inner layer is made of a gel material to provide a soft interface to the skin, optionally with foam, felt or other soft fabric on the inner layer to achieve an even softer interface.

Intermediately on the illustrative device, an anterior portion 118 has an inner wall that defines a chamber 120 to which negative pressure can be applied to preferentially draw tissue away from the center of the throat of the patient, making breathing easier. Though not shown, an air channel or channels may be provided as well on the body of the collar 110 to provide access to the chamber 120 for purposes of applying a vacuum or negative pressure thereto, as well as, optionally, to provide venting to prevent overpressure or to provide a slow air passage in the air chamber to avoid accumulation of sweat, for example, during sleep.

Two structures are shown at 130, 132 that may be used throughout or for a portion of the collar 110 to achieve a semi-rigid structure. In the example shown, a square wave pattern is made of a first resilient material, such as a relative hard plastic or metal, with a softer material coating thereon. In a relaxed state, the pattern takes the form shown at 130. When negative pressure is applied to one side, as shown at 132, the horizontal parts of the pattern are pulled into tension, bringing the vertical parts into closer spacing, making the structure rigid in the vertical direction, while also limiting compression horizontally. The result, in an example, is that the collar section using such a structure (which may be understood as a set of outer circles and inner circles joined together with horizontal segments) becomes stiffer under the negative pressure influence. Such a support structure may be used, but is not necessary to the working of the invention.

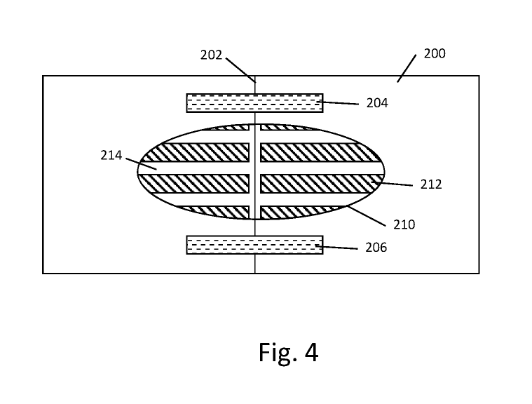

FIG. 4 is a rear view of an illustrative device with partial cut-away. FIG. 4 shows another example of a collar 200. A line of separation is shown at 202 which can be used to allow a patient to apply and remove the collar 200 without having to slide it over the head. To this end, straps are shown as at 204, 206 which may be, for example, Velcro straps to secure the halves of the device on either side of the line of separation 202 to one another. Other securing mechanisms such as a buckle, a clip or locking mechanism, snaps, buttons, a releasable zip-line or ratchet, or adhesive strips may be used instead. In one example, the straps 204, 206 are adjustable to allow the collar fit more closely or more loosely, to match patient comfort. Rather than a strap which extends across, the straps 204, 206 may instead comprise a strap and buckle combination.

The internal structure of this example 200 includes a plurality of reinforcement bands 212, separated by areas of soft material, as indicated in the cutout at 210. For example, the bands 212 may be metal or stiff, resilient plastic, to provide structure to the collar 200, with the soft material 214 provided to make the collar 200 less stiff than it would otherwise be.

In one example, the line of separation may integrate mating pieces, such as pins and holes, to ensure alignment thereof to prevent over-tightening of the collar 200 on the patient's neck, which could lead to discomfort, impairment of blood flow, or even asphyxiation. In an example, the collar 200 is designed to contact the patient only at the upper end, lower end, and rear portions thereof, without contact to the front side of the neck. In other examples, the collar 200 may have a defined area or chamber on the front of the patient's neck, specifically over the trachea, where the chamber (FIG. 3) is defined.

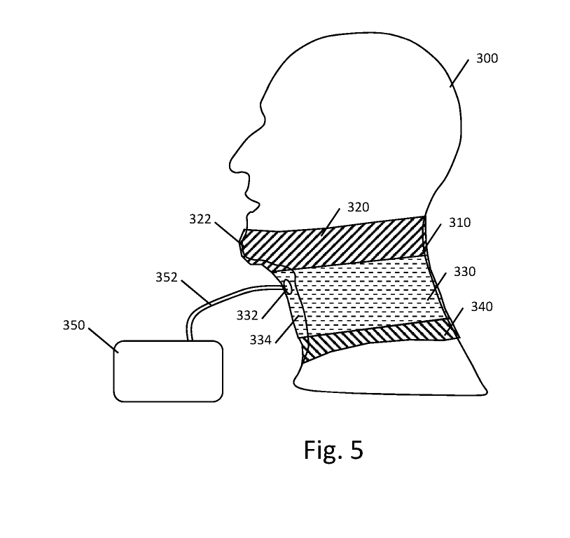

FIG. 5 is a side view of an illustrative system on a patient. In this example, patient 300 has a collar 310 on his/her neck, with an upper first portion 320, an intermediate section 330, and a lower second portion 340. The upper portion 320 is elastic and stretchy, with a soft inner wall adapted to be placed and held against the skin of the patient while providing a seal about the chin 322 of the patient. The lower portion 340 is elastic and stretchy, with a soft inner wall adapted to be placed and held against the skin of the patient while providing a seal about the base of the neck of the patient.

The intermediate portion 330 is stiffer and more resilient than the upper portion 320 and lower portion 340. This stiffness and resilience allows the intermediate portion to define a chamber about the front of the neck, as shown at 334. An air channel 332 is also provided into the chamber 334. In this example, the air channel 332 couples to a port that is adapted to receive a tube 352 from an external air pump 350. The air pump 350 applies negative pressure to the chamber 334 via the tube 352, port, and air channel 332. The negative pressure is used to apply negative pressure on the anterior neck, easing any restriction of the airway due to pressures from the patient's neck tissue, protecting the airway and reducing obstructions that can cause sleep apnea.

In general, known CPAP devices may provide in the range of 30-40 liters per minute of air flow to maintain the patient's internal airway at an elevated pressure to “blow open” the airway and avoid apnea/obstructions. In the present invention, it is anticipated that a good sealing system would only require a few milliliters of air flow per minute to maintain a negative pressure. Depending on the extent and strength of seal, as well as whether some amount of air flow is desired, the negative air pressure may yield a flow rate of up to one liter per minute or less. In other examples, the air flow may be in the range of less than 100 milliliters per minute, or less than 10 milliliters per minute, or in the range of 2-3 milliliters per minute. Total pressure may vary as desired and as is comfortable for a given patient. In an example, the negative pressure may be in the range of 7-12 millimeters water (approximately 70 to 120 Pa). In another example, the total negative pressure is in the range of 5-20 millimeters water (approximately 50 to 200 Pa). Higher or lesser negative pressures may be applied as desired and/or tolerated by the patient. The numeric ranges of this paragraph are merely illustrative; other ranges may also be in the scope of the present invention, and other measures may be used.

If desired, a pressure sensor may be provided to monitor pressure inside the chamber, and if further desired, a pressure release valve may be provided as a way of achieving emergency release (that is a manually actuated valve). In another example, a pressure valve is provided to bleed a small amount of air into the chamber to control maximum pressure while also allowing air circulation which may improve comfort. A wide variety of suitable pumps that can provide quiet and reliable operation are commercially available such as through Rena Aquatics.

In FIG. 5, the vacuum is applied using an external air pump 350. Such a pump may be battery powered or powered using a plug-in to a wall outlet, as desired. An illustrative pump 350 may include circuitry to receive a signal from the collar (such as from a sensor associated with the collar) to detect one or more of pressure, moisture, humidity, temperature, or any other desirable feature (such as using an optical interface to such as a pulse oximeter to determine whether blood flow beneath the collar is pulsatile which would indicate that the pressure applied is not so high as to interfere with such blood flow) to determine whether the pressure beneath the collar is adequate and/or in a desired range. In some examples, a minimum air flow within the collar may be desired for comfort, or airflow within a general range, and pressure, humidity and temperature may be useful, all together or individually, to keep air flow at a desired level. Control circuitry, including for example a microcontroller or state machine, may be provided including machine or computer readable media, which may be non-transitory, containing instructions to monitor pressure or air flow and control the vacuum pump in accordance with preset parameters. A user interface may be provided on pump 350, such as with a screen or analog or digital output, and/or the pump 350 may include wireless communication circuitry such as a Bluetooth™ antenna and/or chip (or any other suitable communication media and circuitry, such as using the Medradio band or other RF), to allow the user 300 to control the pump 350 with a user controller, which may be implemented on a smartphone for example, having its own communication circuitry or used in association with a plug-in controller or dongle.

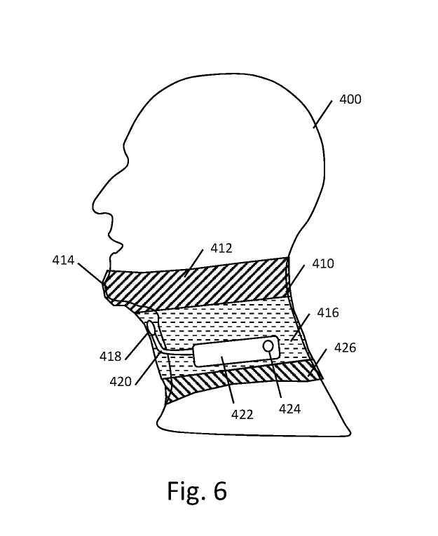

FIG. 6 is a side view of an illustrative integrated device on a patient, in this example, the pump is integrated in the collar. Specifically, patient 400 has a collar 410 having an upper portion 410 over the chin 414, with an intermediate portion 416 defining an air chamber 418 having a port 418 coupled to an air channel 420 that is fluidly connected to an integrated pump 422 having an exit port 424 to allow air to be pumped out of the chamber 418, maintaining negative air pressure on the anterior neck to improve and reduce airway constriction. The pump 422 may be as above in terms of the pressure and air flow provided. The pump 422 is shown mounted in the intermediate portion of the collar 410, with the lower portion 426 provided as above. The pump 422 may instead be on the upper portion 412 or lower portion 426, if desired. The pump 422 may be battery powered, using replaceable or rechargeable batteries, if desired.

An illustrative pump 422 may include circuitry to receive a signal from the collar (such as from a sensor associated with the collar) to detect one or more of pressure, moisture/humidity, temperature, or any other desirable feature (such as using an optical interface to such as a pulse oximeter to determine whether blood flow beneath the collar is pulsatile which would indicate that the pressure applied is not so high as to interfere with such blood flow) to determine whether the pressure beneath the collar is adequate and/or in a desired range. In some examples, a minimum air flow within the collar may be desired for comfort, or airflow within a general range, and pressure, humidity and temperature may be useful, all together or individually, to keep air flow at a desired level. Control circuitry, including for example a microcontroller or state machine, may be provided including machine or computer readable media, which may be non-transitory, containing instructions to monitor pressure or air flow and control the vacuum pump in accordance with preset parameters. A user interface may be provided on pump 422, such as with a screen or analog or digital output, and/or the pump 422 may include wireless communication circuitry such as a Bluetooth™ antenna and/or chip (or any other suitable communication media and circuitry, such as using the Medradio band or other RF), to allow the user 400 to control the pump 422 with a user controller, which may be implemented on a smartphone for example, having its own communication circuitry or used in association with a plug-in controller or dongle.

As may be appreciated by the skilled artisan, the upper end 412 of the collar 410 abuts and, in the example shown, encircles a portion of the jaw at 414. The more rigid intermediate portion 416 may physically improve airway management by preventing the jaw from dropping down in the region of the neck and constricting airflow. The soft palate may be supported by the upper end 412 of the collar 410, for example, as an adjunct to the vacuum that is provided at chamber 418. Such structural support, when combined with the positive atmospheric pressure seen by the nasopharynx, may also keep the soft palate in contact with the tongue, helping to keep both tongue and soft palate from obstructing the airway.

In an example, the collars as shown above may be manufactured by obtaining an image of the patient's neck, preferably with sufficient views to obtain a 360 degree view thereof with surface contours, to allow a collar to be manufactured by additive processes such as by 3D printing the collar. During such 3D printing, different areas of the device may be formed using different materials to achieve softer portions and harder or more rigid portions. In other examples, the rigid structure, such as bands shown above, may be formed separately and added to a 3D printed soft structure, where the softer structure is specifically configured for a given patient. In another example, the intermediate portion (FIGS. 5-6) may be a stock item or may come in a handful of sizes, while the upper and lower portions may be 3D printed or otherwise tailored to the given patient to account for the wide variety of jawlines and upper thorax structures of the patient population. In still other examples, the collar may come in a range of sizes and shapes for particular patients, where pre-use imaging may or may not be used to select a collar for a given patient.

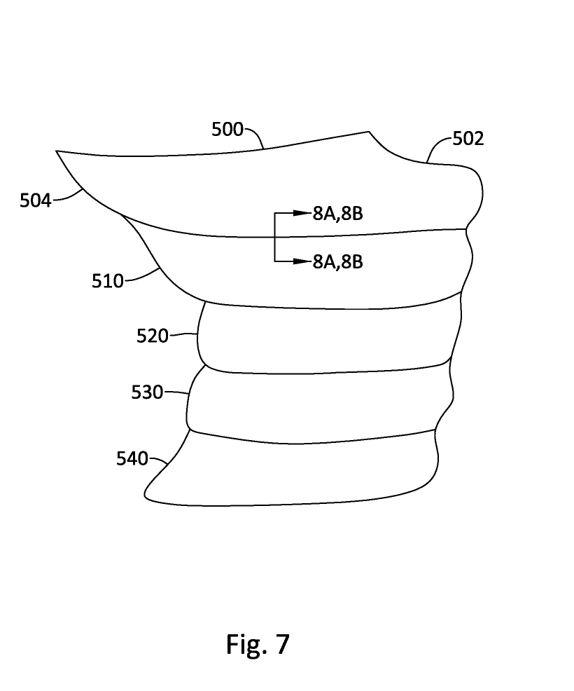

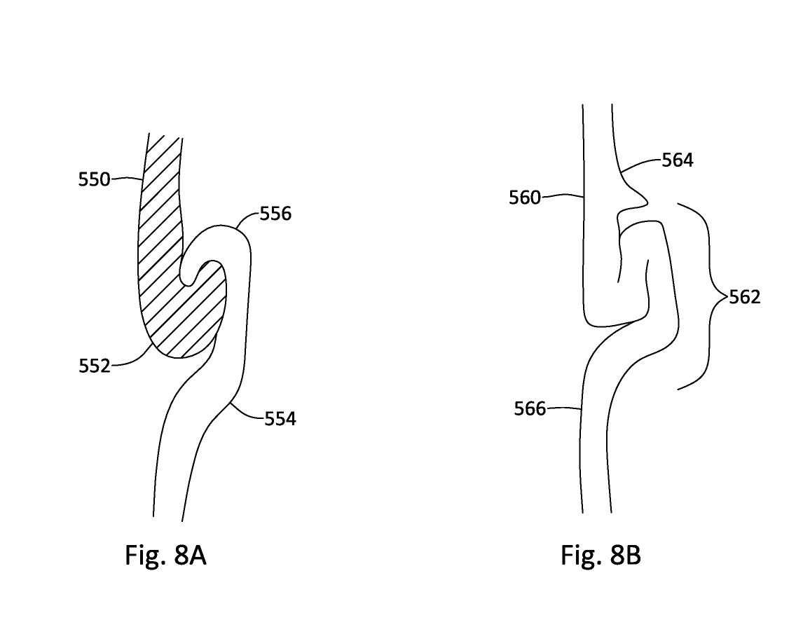

FIG. 7 is a side view of an illustrative device construction. Here, a plurality of interlocking rings are provided up and down on the device, which would allow for movement in the vertical direction as well as some twisting motion, while providing rigidity to offer a chamber that can apply negative pressure on the anterior neck. In the example, the collar internal structure is shown at 500, with a cutout at 502 where the structure would interact with the skull (to avoid rubbing over bony tissue), with a first portion extending along the jawline as indicated at 504. Interlocking rings are provided at 510, 520, 530, 540, having joints as shown below in FIGS. 8A-8B. The interlocking rings as shown would be covered on at least the interior side thereof with an inner lining adapted for comfort and sealing properties, such as using silicone at the tissue interface and a non-latex rubber or elastomer or other flexible, biocompatible polymer, for example, to define the hermetically sealed chamber as well as tissue interface surfaces.

FIGS. 8A-8B show a partial section view of a joint between layers of the embodiment of FIG. 7. In an example shown at FIG. 8A, a first ring 550 couples to a second ring 554 at a joint defined by hooked portions 552, 556, that interact to provide a secure yet flexible attachment thereof. In an example shown at FIG. 8B, a single piece structure 560 with a joint formed by imparting a set of curves as shown at 562 between a first ring 564 and a second ring 566.

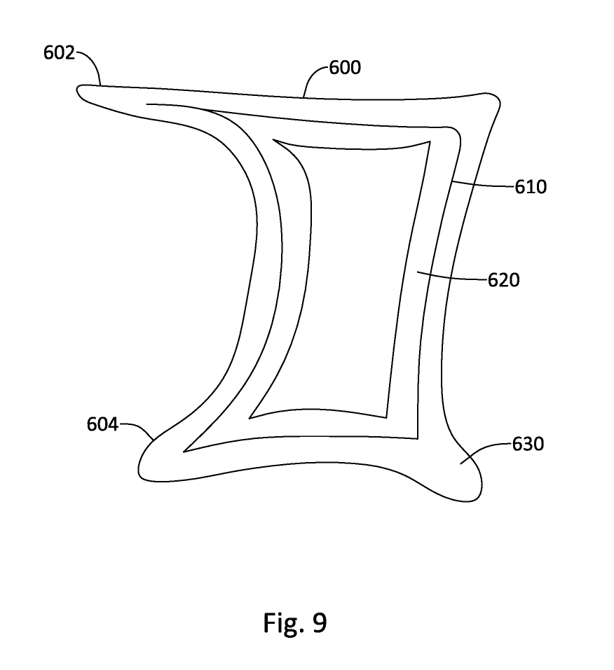

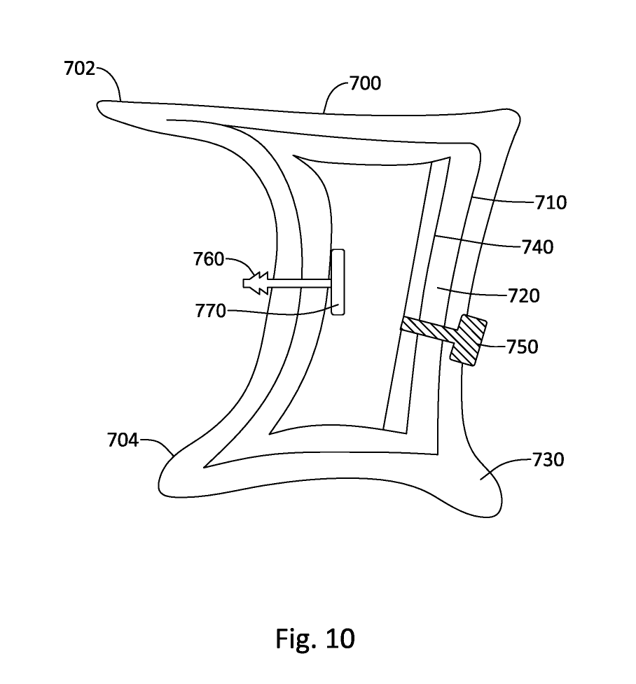

FIGS. 9-10 show a medial slice of each of two illustrative examples. As shown in FIG. 9, the collar 600 has an upper portion with jaw interface at 602, and a chest/base interface at 604. The overall structure has a thin, relatively strong or rigid core layer shown at 610, with soft outer layer 630 and soft inner layer 620. The core layer 610 may be of a rigid plastic to maintain overall shape of the collar 600. The inner and outer layers may be any suitable material or may even have more than one layer of material using, for example, a non-latex rubber or a soft polymer, or even silicone and/or silicone gel.

As shown in FIG. 10, another alternative collar 700 has an upper portion with jaw interface at 702, and a chest/base interface at 704. The overall structure has a thin, relatively strong or rigid core layer shown at 710, with soft outer layer 730 and soft inner layer 720. The core layer 710 may be of a rigid plastic to maintain overall shape of the collar 700. The inner and outer layers may be any suitable material or may even have more than one layer of material using, for example, a non-latex rubber or a soft polymer, or even silicone and/or silicone gel.

Further in FIG. 10, an additional inner layer is provided on the tissue contacting back side, as shown at 740. This layer may be a replaceable component that is adapted to contact the back of the patient's neck. Over time, the tissue interface may become soiled and therefore can be removed and replaced to ensure continued comfort and to, for example, avoid smells and discoloration that can be caused by sweat and oils generated during use. A leak valve is shown at 750, and may be used as described above to allow a small amount of air leakage into the chamber defined by the collar 700, and a port is shown at 760 to allow a tube that in turn attaches to an air pump to be attached, in order to apply a vacuum to the chamber defined by the anterior portion of the collar. The air pump may be as in any of the above preceding embodiments.

The device may further include a grid or piece of spongy material 770 to allow the vacuum to be applied in a dispersed manner, preventing the patient's skin (which may be loose for some older or obese patients) from being sucked into the vacuum port. Element 770 may also be a replaceable component of the collar.

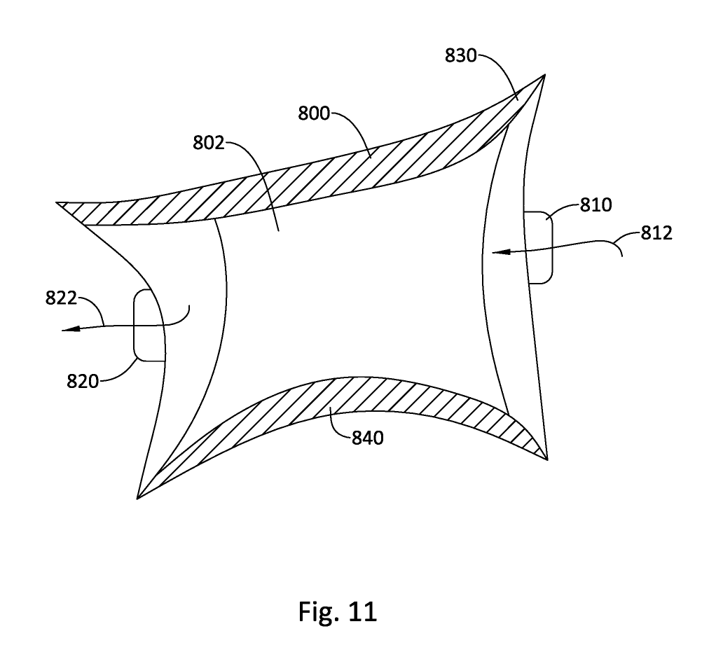

FIG. 11 is a side view of another illustrative device. The collar 800 is placed on the neck 802 of a patient. As can be seen, the device has a posteriorly placed inflow valve at 810 to allow controlled air flow indicated at 812 into the interior of the collar 800, while a pump 820 draws air out as shown at 822. As noted above, such a construction allows air to circulate, possibly improving comfort. Air circulation may be constant or periodic such as in response to sensed pressure conditions, humidity, or at elapsed time intervals, as desired. A foam, gel, or otherwise soft interface is provided at the upper end 830, as well as at the lower end 840, of the collar, to provide comfort and a semi-airtight, or fully hermetic, seal at the upper and lower ends of the collar 800.

An illustrative valve 810 and/or pump 820 may include circuitry to receive a signal from the collar (such as from a sensor associated with the collar) to detect one or more of pressure, moisture/humidity, temperature, or any other desirable feature (such as using an optical interface to such as a pulse oximeter to determine whether blood flow beneath the collar is pulsatile which would indicate that the pressure applied is not so high as to interfere with such blood flow) to determine whether the pressure beneath the collar is adequate and/or in a desired range. For the more integrated example of FIG. 11, having the valve 810 and pump 820 as part of the collar itself, such sensors may be in or on the valve and/or pump 820. The valve 810 and pump 820 may be electrically connected (via one or more wires, for example) or may communicate using a wireless protocol (such as Bluetooth™ Low Energy), or may operate independently. In some examples, a minimum air flow within the collar may be desired for comfort, or airflow within a general range, and pressure, humidity and temperature may be useful, all together or individually, to keep air flow at a desired level. Control circuitry, including for example a microcontroller or state machine, may be provided including machine or computer readable media, which may be non-transitory, containing instructions to monitor pressure or air flow and control the vacuum pump in accordance with preset parameters, in one, the other, or both of the valve 810 and/or pump 820. The valve 810 and/or pump 820 may include wireless communication circuitry such as a Bluetooth™ antenna and/or chip (or any other suitable communication media and circuitry, such as using the Medradio band or other RF), to allow the user to control the valve 810 and/or pump 820 with a user controller, which may be implemented on a smartphone for example, having its own communication circuitry or used in association with a plug-in controller or dongle. Alternatively, a button or buttons may be provided on the collar 800 and/or the valve 810 and/or pump 820 to activate or change parameters, such as the degree of negative pressure applied by the system.

While the system is discussed herein primarily in the context of a device to treat sleep apnea, an acute trauma device may also be realized using similar principals. While tailoring to a particular patient by the use of imaging and additive manufacturing, or casting and molding processes discussed below may not be feasible for an acute case, the application of a collar about the neck of a patient as disclosed herein may be useful to help maintain an open airway in the event a breathing tube, for whatever reasons, cannot be used, is unavailable, or is contraindicated.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description.

The Abstract is provided to comply with 37 C.F.R. § 1.72(b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A collar for the treatment of sleep apnea comprising:
a first end having a portion made of a soft, flexible material to provide a seal against the skin of the patient near the chin;
a second end having a portion made of a soft, flexible material to provide a seal against the skin of the patient near the base of the neck; and
a body between the first and second ends, the body having an inner wall for defining a chamber about the neck of the patient and at least one air channel associated with a first port in the inner wall.

2. The collar of claim 1 wherein the first and second ends are configured such that the chamber is generally airtight to facilitate application of negative pressure to the neck of the patient to pull soft tissue away from the center of the throat, allowing the patient to breathe.

3. The collar of claim 1 wherein at least one of the first or second ends is made of silicone or a soft gel to facilitate patient comfort and a seal against the skin of the patient.

4. The collar of claim 1 wherein the body comprises an inner layer of relatively more rigid material to maintain the chamber.

5. The collar of claim 1 wherein the first end is configured to push open the soft palate by limiting movement of the jaw of the patient.

6. The collar of claim 1 wherein the body includes a back portion having a line of separation allowing the collar to be removed from and/or applied to the neck of a patient, and a front portion in which the first port is located.

7. The collar of claim 1 further comprising a vent and a pressure sensor, the vent coupled to the air channel to allow release of pressure therein, wherein the pressure sensor is adapted to sense pressure in the chamber and/or air channel and open the vent to prevent over-pressurization.

8. The collar of claim 1 further comprising a leak valve coupled to the inner wall and adapted to limit pressurization of the chamber.

9. The collar of claim 1 wherein the body comprises a coiled support structure to allow longitudinal flexibility between the first and second ends, and lateral support to maintain the chamber.

10. The collar of claim 1 wherein the body comprises a plurality of support structures in the form of rings, the rings being interrupted at the location of the line of separation.

11. A system comprising the collar of claim 1 and an external vacuum motor coupled by a tube to a second port of the air channel.

12. A collar as in claim 1 further comprising a vacuum motor coupled to a second port of the air channel for applying a vacuum to the chamber.

13. A method of treating a patient having a breathing disorder comprising:
applying a collar to the patient's neck; and
applying negative air pressure to the patient's neck via the collar.

14. The method of claim 13 wherein the breathing disorder is obstructive sleep apnea.

15. The method of claim 13 wherein the step of applying negative air pressure comprises coupling a vacuum pump to the collar, and activating the vacuum pump.

16. The method of claim 13 wherein the collar comprises a leak valve coupled to the collar which is adapted to limit the negative air pressure applied to the patient's neck.

17. The method of claim 13 wherein the collar comprises:
a first end having a portion made of a soft, flexible material to provide a seal against the skin of the patient near the chin;
a second end having a portion made of a soft, flexible material to provide a seal against the skin of the patient near the base of the neck; and
a body between the first and second ends, the body having an inner wall for defining a chamber about the neck of the patient and at least one air channel associated with a first port in the inner wall;
such that the step of applying negative air pressure comprises reducing the air pressure within the defined chamber.

18. The method of claim 17 wherein the first and second ends of the collar are configured such that the chamber is generally airtight to facilitate application of negative pressure to the neck of the patient, such that the step of applying negative air pressure causes pulling of soft tissue away from the center of the throat, thereby allowing the patient to breathe.

19. A method of treating a patient comprising making a collar to prevent sleep apnea by:
obtaining one or more images of a region of the patient including the neck; and
making a collar for treating sleep apnea using the obtained images to construct a collar having an upper end to provide an airtight and comfortable seal at the upper neck and/or jaw of the patient and a lower end to provide an airtight and comfortable seal at the base of the neck of the patient, with an intermediate section therebetween defining a chamber for applying negative pressure to the patient's anterior neck.
